(19) United States
(12) Patent Application Publication
(10) Pub. No.: US 2005/0111617 A1
(43) Pub. Date: May 26, 2005

(54) Mammography apparatus and detecting unit

(75) Inventor: Takashi Shoji, Kanagawa-ken (JP)

Correspondence Address: SUGHRUE MION, PLLC, 2100 PENNSYLVANIA AVENUE, N.W., SUITE 800, WASHINGTON, DC, 20037, US

(73) Assignee: FUJI PHOTO FILM CO., LTD.

(21) Appl. No.: 10/968,073

(22) Filed: Oct. 20, 2004

(30) Foreign Application Priority Data
Nov. 24, 2003 (JP) (PATENT)364333/20

(51) Int. Cl. A61B 6/04; G01J 1/00; G01T 1/24

(52) U.S. Cl. 378/37

(57) ABSTRACT

Detection accuracy of X-ray dosages, to be applied to control the amount of irradiated X-rays, is improved in a mammography apparatus that employs solid state detectors as X-ray image detecting means, and that is capable of interchangeably utilizing solid state detectors of various sizes, without adversely influencing detection of images by the solid state detector. A solid state detector, for recording image information by receiving irradiation of X-rays that bear the image information, and for outputting image signals that represent the recorded image information, an X-ray dosage detector, for detecting the amount of irradiated X-rays, and a moving grid, for removing scattered radiation, are housed within a detecting unit, stacked in this order. The detecting unit is configured to be removably attachable to an imaging table of a mammography apparatus. Thereby, interchangeable use of detecting units having solid state detectors of various sizes housed therein is enabled.

### BACKGROUND OF THE INVENTION

1. Field of the Invention

The present invention relates to a detecting unit that houses therein a solid state detector, and to a mammography apparatus that obtains X-ray images of breasts by utilizing the detecting unit.

2. Description of the Related Art

Presently, various X-ray imaging apparatuses, for obtaining X-ray images to be utilized for medical diagnoses, have been proposed and are in practical use. These X-ray imaging apparatuses employ solid state detectors (having semiconductors as main components thereof) as X-ray image detecting means. The solid state detectors detect X-rays that have passed through subjects, and obtain image signals that represent X-ray images of the subject.

A variety of formats have been proposed for the solid state detectors to be utilized in these apparatuses. Regarding a charge generating process for converting X-rays to electrical charges, there is a photo conversion type of solid state detector, and a direct conversion type of solid state detector, for example. The photo conversion type of solid state detector temporarily stores signal charges, obtained at a photoconductive layer by detecting fluorescence emitted by phosphors due to irradiation with X-rays, in a charge accumulating portion, then converts the accumulated charges to image signals (electrical signals) and outputs the image signals. The direct conversion type of solid state detector temporarily stores signal charges, generated within a photoconductive layer due to irradiation with X-rays and collected by a charge collecting electrode, in a charge accumulating portion, then converts the accumulated charges to electric signals and outputs the electric signals. In this type of solid state detector, the main components are the photoconductive layer and the charge collecting electrode.

Regarding a charge readout process for reading out the accumulated charges, there are an optical readout technique and a TFT readout technique. In the optical readout technique, accumulated charges are read out by irradiating a solid state detector with readout light (electromagnetic waves for readout). In the TFT readout technique, accumulated charges are read out by scanning TFT's (thin film transistors), which are connected to a charge accumulating portion. The TFT readout technique is disclosed in Japanese Unexamined Patent Publication No. 2000-244824.

An improved direct conversion type solid state detector has also been proposed in U.S. Pat. No. 6,268,614. The improved direct conversion type solid state detector is a direct conversion type of solid state detector that utilizes the optical readout technique. This solid state detector comprises: a recording photoconductive layer that exhibits photoconductivity when irradiated by recording light (X-rays, or fluorescence generated by the irradiation of X-rays); a charge transport layer that acts substantially as an insulator with respect to charges having the same polarity as latent image charges, and that acts substantially as a conductor with respect to charges having the opposite polarity as latent image charges; and a readout photoconductive layer that exhibits photoconductivity when irradiated by electromagnetic waves for readout; stacked in this order. Signal charges (latent image charges) that bear image information are accumulated at an interface (charge accumulating portion) between the recording photoconductive layer and the charge transport layer. Electrodes (a first conductive layer and a second conductive layer) are provided on both sides of the three aforementioned layers. In the solid state detector having this format, the recording photoconductive layer, the charge transport layer, and the readout photoconductive layer are the main components.

Conventional mammography apparatuses employ imaging plates, film, and the like as X-ray image detecting means. In these conventional apparatuses, the X-ray image detecting means is housed within a detecting unit, and removably held on an imaging table of the mammography apparatus. X-ray image detecting means of various sizes (for example, 18 cm×24 cm, 24 cm×30 cm, etc.) are interchangeably usable in these apparatuses. However, no mammography apparatus has been disclosed to date that enables solid state detectors of various sizes to be utilized interchangeably.

During X-ray imaging, it is necessary to appropriately control the amount of X-rays irradiated onto the X-ray image detecting means, in order to obtain an optimal image. Therefore, apparatuses have been proposed, in which an X-ray dosage detector detects the amount of X-rays irradiated onto the X-ray image detecting means, and the detected X-ray dosage is applied to the control of the amount of emitted X-rays. However, in these apparatuses, the X-ray dosage detector is housed within the imaging table. Therefore, if the X-ray absorption rate of the X-ray image detecting means is increased to improve image quality, the amount of X-rays that pass through the X-ray image detecting means decreases. Consequently, a problem arises in that it becomes difficult for the X-ray dosage detector to detect the X-ray dosage accurately. In order to solve this problem, the X-ray dosage detector may be provided above the X-ray image detecting means as a separate unit. However, in this case, the distance between subjects and the X-ray image detecting means becomes great, and the resolution of images detected thereby decreases.

That is, no mammography apparatus has been disclosed to date that employs solid state detectors as the X-ray image detecting means, in which solid state detectors of various sizes are interchangeably usable and which enables accurate detection of X-ray dosages, to be applied to control the amount of irradiated X-rays, without adversely influencing detection of images by the solid state detectors.

### SUMMARY OF THE INVENTION

The present invention has been developed in view of the above circumstances. It is an object of the present invention to provide a mammography apparatus that employs solid state detectors as X-ray image detecting means, in which solid state detectors of various sizes are interchangeably usable, and in which accuracy in detecting X-ray dosages, to be applied to control the amount of irradiated X-rays, is improved without adversely influencing detection of images by the solid state detectors.

The mammography apparatus according to the present invention comprises:

a detecting unit constituted by a solid state detector for receiving irradiation of X-rays that bear image information to record the image information, and for outputting image signals that represent the recorded image information, and an X-ray dosage detector, which is provided between a radiation source of the X-rays and the solid state detector, for detecting the dosage of the irradiated X-rays; and

a holding portion for removably holding the detecting unit.

It is preferable that the detecting unit of the mammography apparatus according to the present invention further comprises:

a grid, for removing scattered radiation, provided between the radiation source of the X-rays and the solid state detector. Note that the grid is to be placed between the X-ray source and the solid state detector, and may be provided between the X-ray source and the X-ray dosage detector, or between the X-ray dosage detector and the solid state detector.

The detecting unit according to the present invention comprises:

a solid state detector for receiving irradiation of X-rays that bear image information to record the image information, and for outputting image signals that represent the recorded image information;

and an X-ray dosage detector, which is provided between a radiation source of the X-rays and the solid state detector, for detecting the dosage of the irradiated X-rays.

It is preferable that the detecting unit further comprises:

a grid, for removing scattered radiation, provided between the radiation source of the X-rays and the solid state detector. Note that the grid is to be placed between the X-ray source and the solid state detector, and may be provided between the X-ray source and the X-ray dosage detector, or between the X-ray dosage detector and the solid state detector.

In the present invention, “solid state detectors” refer to detectors that detect X-rays that bear image information of subjects, and output image signals that represent X-ray images of the subjects. The solid state detectors convert X-rays incident thereon to electrical charges, either directly or after converting the X-rays to light. Image signals representing the X-ray images of the subjects are obtained by outputting these electrical charges.

There are a variety of formats for the solid state detector. Regarding a charge generating process for converting X-rays to electrical charges, there is a photo conversion type of solid state detector, and a direct conversion type of solid state detector, for example. The photo conversion type of solid state detector temporarily stores signal charges, obtained at a photoconductive layer by detecting fluorescence emitted by phosphors due to irradiation with X-rays, in a charge accumulating portion, then converts the accumulated charges to image signals (electrical signals) and outputs the image signals. The direct conversion type of solid state detector temporarily stores signal charges, generated within a photoconductive layer due to irradiation with radiation and collected by a charge collecting electrode, in a charge accumulating portion, then converts the accumulated charges to electric signals and outputs the electric signals. Regarding a charge readout process for reading out the accumulated charges, there are an optical readout technique and a TFT readout technique. In the optical readout technique, accumulated charges are read out by irradiating a solid state detector with readout light (electromagnetic waves for readout). In the TFT readout technique, accumulated charges are read out by scanning TFT's (thin film transistors), which are connected to a charge accumulating portion. Further, there are solid state detectors that combine the direct conversion type and the optical readout method, such as the improved direct conversion type solid state detector, as disclosed in U.S. Pat. No. 6,268,614.

The mammography apparatus according to the present invention comprises: a detecting unit constituted by a solid state detector for recording image information by receiving irradiation of X-rays that bear the image information and for outputting image signals that represent the recorded image information, and an X-ray dosage detector for detecting the amount of irradiated X-rays, provided between an X-ray source and the solid state detector; and a holding portion for removably holding the detecting unit. Therefore, detecting units equipped with solid state detectors of various sizes can be interchangeably utilized. In addition, by providing the X-ray dosage detector between the X-ray source and the solid state detector, accurate measurement of the X-ray dosage can be performed without influence from the solid state detector.

A configuration may be adopted wherein the detecting unit further comprises a grid, for removing scattered radiation, provided between the radiation source of the X-rays and the solid state detector. In this case, the image quality of images detected by the solid state detector can be improved.

The detecting unit according to the present invention comprises: a solid state detector for receiving irradiation of X-rays that bear image information to record the image information, and for outputting image signals that represent the recorded image information; and an X-ray dosage detector, which is provided between a radiation source of the X-rays and the solid state detector, for detecting the dosage of the irradiated X-rays. By configuring the detecting unit to be removably attachable to a holding portion of a mammography apparatus, solid state detectors of various sizes are enabled to be interchangeably used by the mammography apparatus.

A configuration may be adopted wherein the detecting unit further comprises a grid, for removing scattered radiation, provided between the radiation source of the X-rays and the solid state detector. In this case, the image quality of images detected by the solid state detector can be improved.

### BRIEF DESCRIPTION OF THE DRAWINGS

FIG. 1 is a schematic diagram illustrating an example of a mammography apparatus, to which the X-ray imaging device according to the present invention is applied.

FIG. 2 is a schematic diagram illustrating the interior of a film cassette of the mammography apparatus.

FIG. 3 is a schematic diagram illustrating a conductive layer portion of an X-ray dosage detector of the mammography apparatus.

FIG. 4 is a circuit diagram illustrating an integrating circuit and a comparative circuit of the mammography apparatus.

FIG. 5 is a timing chart of each operation of the mammography apparatus, from imaging to readout.

### DESCRIPTION OF THE PREFERRED EMBODIMENTS

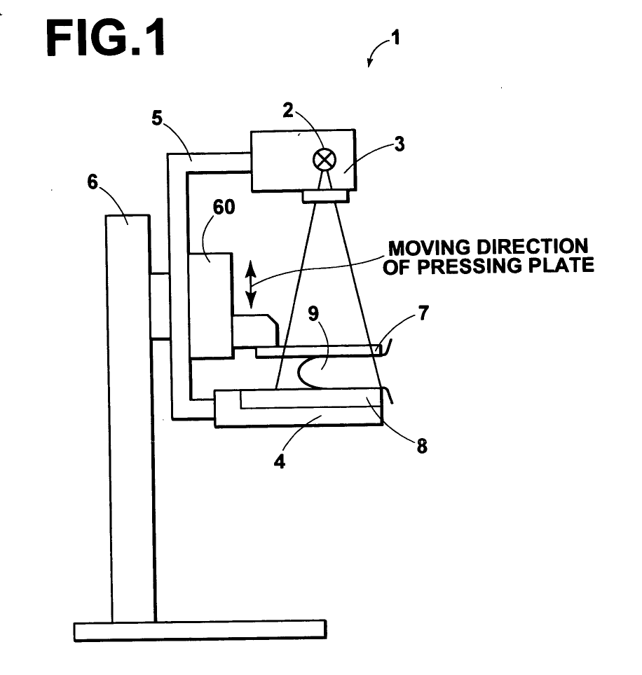

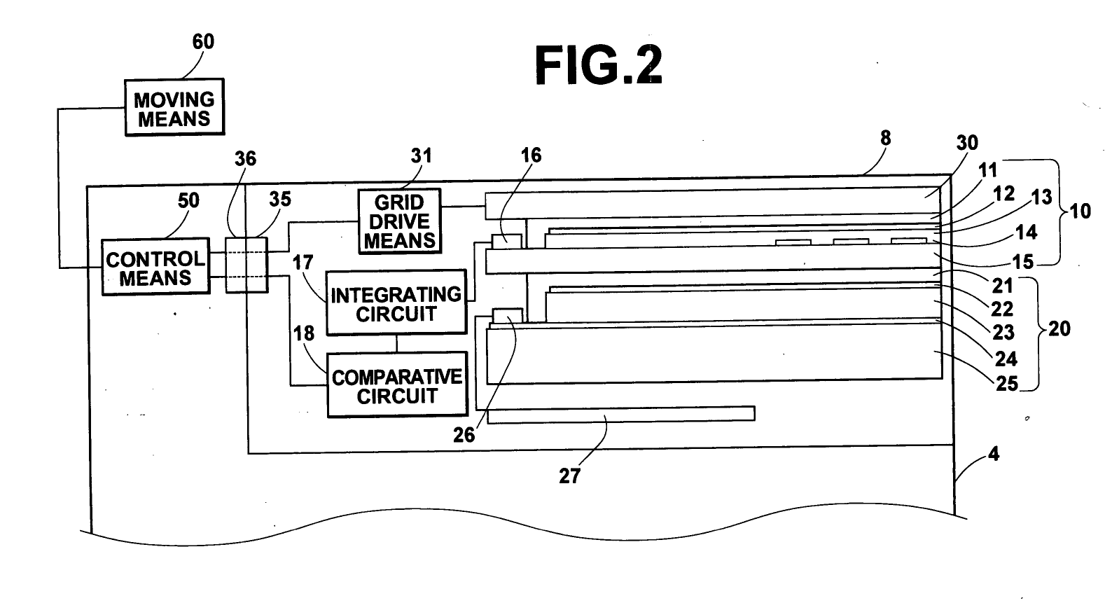

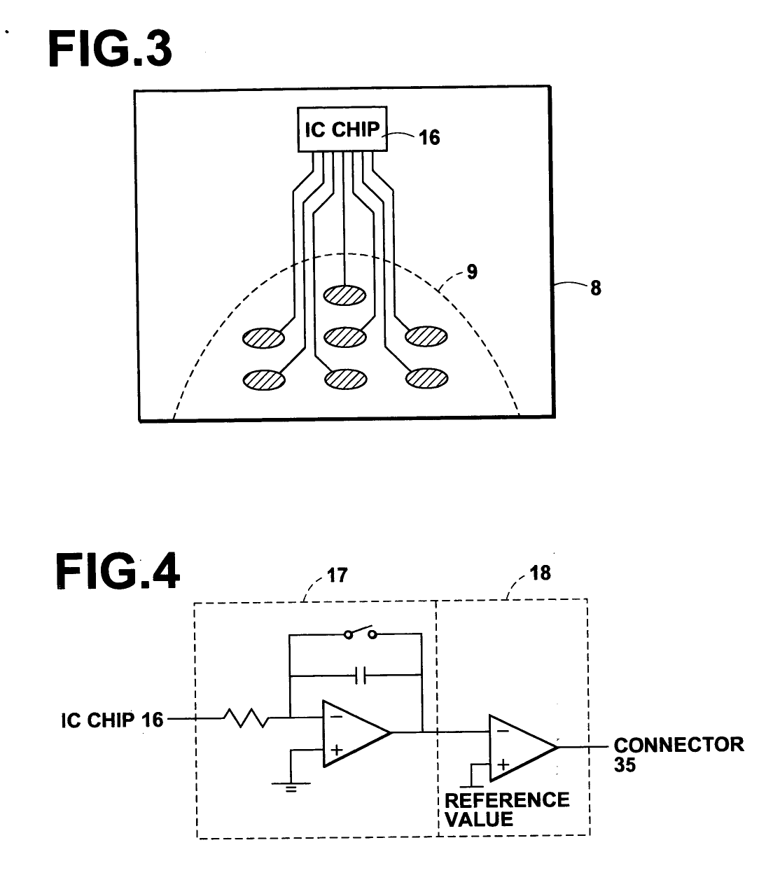

Hereinafter, an embodiment of the present invention will be described in detail with reference to the attached drawings. FIG. 1 is a schematic diagram that illustrates an example of a mammography apparatus according to the present invention. FIG. 2 is a schematic diagram that illustrates the interior of a detecting unit of the mammography apparatus. FIG. 3 is a schematic diagram illustrating a conductive layer portion of an X-ray dosage detector of the mammography apparatus. FIG. 4 is a circuit diagram illustrating an integrating circuit and a comparative circuit of the mammography apparatus.

A mammography apparatus 1 comprises: an X-ray source housing portion 3 that houses an X-ray source 2 within its interior; an imaging table 4 for holding a detecting unit 8; arms 5; and a base 6. The X-ray source housing portion 3 and the imaging table 4 are linked by the arms 5 so that they face each other. The arms are mounted on the base 6.

Further, a pressing plate 7, for pressing and holding a subject's breast 9 from above, and a pressing plate moving means 60, for moving the pressing plate 7 automatically in response to commands from a control means 50, are mounted on the arms 5. The pressing plate moving means 60 is constituted by a linear motor (not shown). The pressing plate moving means 60 moves the pressing plate 7 reciprocally between a first position, at which the breast 9 is pressed against the detecting unit 8 held on the imaging table 4, and a second position, at which the pressure is released.

An X-ray dosage detector 10, for detecting the dosage of X-rays irradiated on the detecting unit 8; a solid state detector 20, which is an imaging device; a moving grid 30, for removing scattered radiation; a grid drive means 31, for driving the moving grid 30; and a power source (not shown), for supplying electricity to the above components, are provided within the detecting unit 8.

The X-ray dosage detector comprises: a first conductive layer 14; a photoconductive layer 13 that generates electrical charges and exhibits conductivity when irradiated with X-rays; a second conductive layer 12; and an insulative layer 11, stacked in this order on a resin substrate 15.

The first conductive layer 14 comprises a plurality of conductive layer portions 14a, which are formed separated from each other, as illustrated in FIG. 3. Each of the conductive layer portions 14a is connected to an IC chip 16. Further, the IC chip 16 is connected to an integrating circuit 17, and the integrating circuit 17 is connected to a comparative circuit 18.

The X-ray dosage detector 10 operates in the following manner. Electric fields are formed between each of the conductive layer portions 14a of the first conductive layer 14 and the second conductive layer 12. If X-rays are irradiated onto the photoconductive layer 13 at this time, charge pairs are generated within the photoconductive layer 13. Current corresponding to the amount of charge pairs flows between each conductive layer portion 14a and the second conductive layer 12, and the current is converted to voltage by the IC chip 16.

The integrating circuit 17 converts the current that flows between each conductive layer portion 14a and the second conductive layer 12 into voltages, and integrates the converted voltages. In the case that the voltages integrated by the integrating circuit 17 exceed a predetermined value, the comparative circuit 18 outputs data indicating this fact. Thereby, judgment can be made regarding whether the X-ray dosage irradiated on the film cassette 8 has exceeded a predetermined value.

Note that the judgment regarding whether the X-ray dosage irradiated on the film cassette 8 has exceeded the predetermined value may be made based on the current that flows between any one of the plurality of conductive layer portions 14a and the second conductive layer 12, or based on the total current that flows between each of the plurality of conductive layer portions 14a and the second conductive layer 12.

The solid state detector 20 comprises: a first conductive layer 24 formed of a—Si TFT's; a photoconductive layer 23 that exhibits conductivity by generating charges when irradiated with X-rays; a second conductive layer 22; and an insulative layer 21, which are stacked in this order on a glass substrate 25.

A TFT is formed corresponding to each pixel in the first conductive layer 24. Output from each TFT is connected to an IC chip 26, and the IC chip 26 is connected to a printed circuit board 27, which is equipped with an A/D converting portion, a memory, and the like (not shown).

The solid state detector 20 operates in the following manner. An electric field is formed between the first conductive layer 24 and the second conductive layer 22. If X-rays are irradiated onto the photoconductive layer 23 at this time, charge pairs are generated within the photoconductive layer 23. Latent image charges corresponding to the amount of charge pairs are accumulated within the first conductive layer 24. When reading out the accumulated latent image charges, the TFT's of the first conductive layer 24 are sequentially driven to read out the latent image charges corresponding to each pixel. Thereby, an electrostatic latent image borne by the latent image charges are read out.

The aforementioned X-ray dosage detector 10 is stacked on top of the solid state detector 20, and configured to be positioned between the X-ray source 2 and the solid state detector 20 when the film cassette 8 is held on the imaging table 4. For this reason, the X-ray dosage detector 10 is capable of directly detecting the X-rays emitted from the X-ray source, without the solid state detector 20 acting as an intermediary. Therefore, the X-ray dosage can be accurately measured, without being influenced by the solid state detector 20. In addition, because the X-ray dosage detector 10 is formed on a resin substrate 15, which has a lower X-ray absorption rate than glass substrates, adverse influences on detection of X-ray images by the solid state detector 20 are reduced. Accordingly, the image quality of images detected by the solid state detector 20 is improved. Note that a carbon plate or aluminum oxides may be employed as the material of the substrate, as alternatives to resin.

Here, a description will be given of the photoconductive layer 13 and the photoconductive layer 23, which are employed in the X-ray dosage detector 10 and the solid state detector 20, respectively.

The X-ray spectra commonly emitted from X-ray sources is not uniform at all X-ray energies. The X-ray absorption coefficient differs for X-ray energies, depending on the material that constitutes a photoconductive layer. For these reasons, in the case that the photoconductive layer 13 of the X-ray dosage detector 10 and the photoconductive layer 23 of the solid state detector 20 are formed by different materials, there is a possibility that the spectrum of the X-rays, which pass through the X-ray dosage detector 10 and which are detected by the solid state detector 20, will change drastically within the X-ray dosage detector 10. If such a change in the X-ray spectrum occurs, there is a possibility that adverse influences will be imparted on the detection of X-ray images by the solid state detector 20.

Therefore, in the present embodiment, both the photoconductive layer 13 of the X-ray dosage detector 10 and the photoconductive layer 23 of the solid state detector 20 are constituted by a—Se. Thereby, adverse influences imparted on the detection of X-ray images by the solid state detector 20 are reduced, and the image quality of images detected by the solid state detector 20 is improved.

The mammography apparatus 1 comprises the control means 50, for controlling the X-ray source 2, the pressing plate moving means 60, the grid drive means 31 and the like. The detecting unit 8 is equipped with a connector 35, for engaging a connector 36, which is provided on the imaging table 4. The grid drive means 31 and the comparative circuit 18 are connected to the control means 50 via the connectors 35 and 36, while the detecting unit 8 is held on the imaging table 4.

By adopting the construction described above, it becomes possible to removably attach the detecting unit 8 to the imaging table 4. Therefore, detecting units 8 that house solid state detectors 20 of various sizes, corresponding to the size of a subject's breast, can be interchangeably used.

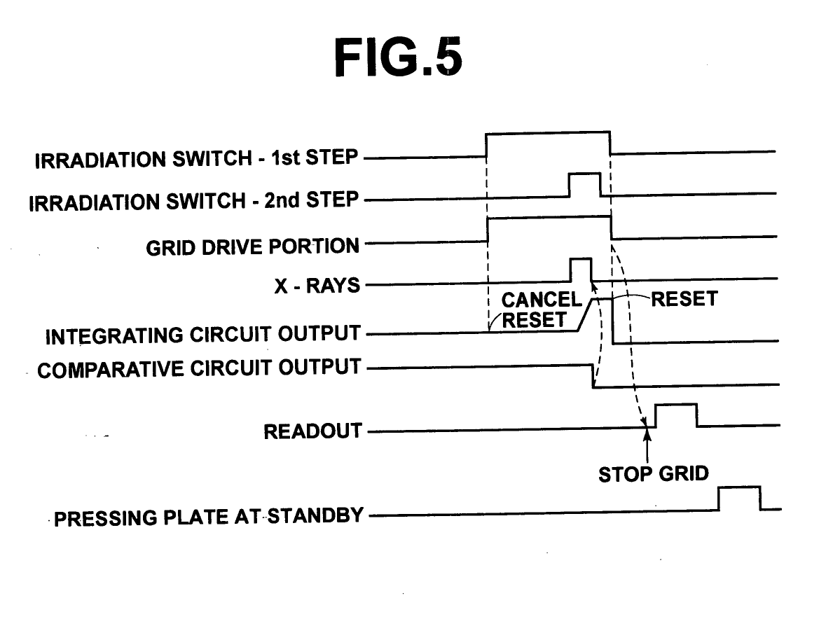

Next, the operation of the mammography apparatus 1, which is constructed as described above, will be described. FIG. 5 is a timing chart of each operation of the mammography apparatus, from imaging to readout.

During imaging, the control means drives the pressing plate moving means 60 to move the pressing plate 7 to the first position at which a breast 9 is pressed, based on commands which are manually input by an operator. Thereby, the breast 9 is fixed on the film cassette 8.

Next, the operator presses a first step of a two step irradiation switch (not shown), and the control means 50 causes the grid drive means 31 to drive the moving grid 30 and cancels resetting of the integrating circuit 17.

Thereafter, the operator presses the second step of the irradiation switch, and the control means 50 causes the X-ray source 2 to emit X-rays onto the breast 9. The X-rays, which have passed through the breast 9, that is, the X-rays that bear X-ray image information of the breast 9, are irradiated on the detecting unit 8. These X-rays are detected by each of the conductive layer portions 14a of the X-ray dosage detector, and voltages corresponding to the X-ray dosage are integrated by the integrating circuit 17. Latent image charges that bear the X-ray image information are accumulated within the solid state detector 20. The amount of accumulated latent image charges is substantially proportional to the X-ray dosage which has passed through a subject. Therefore, the latent image charges bear the electrostatic latent image.

If the output of the integrating circuit, that is, the dosage of X-rays irradiated on the detecting unit 8, exceeds a predetermined value, information indicating this fact is transmitted from the comparative circuit 18 to the control means 50, and the control means 50 stops the X-ray source when this information is received.

When the first step of the irradiation switch is released by the operator, the control means 50 causes the grid drive means 31 to stop the moving grid 30, resets the integrating circuit, and performs readout of the latent image charges from the solid state detector 20.

After readout of the latent image charges is completed, the control means drives the pressing plate moving means 60 to move the pressing plate 7 to the second position, at which the pressure on the breast .9 is released, and the process ends.

Noise becomes overlapped with the latent image charges, if vibration is imparted to the solid state detector 20 during readout of the latent image charges therefrom. However, this problem can be overcome by reading out the latent image charges from the solid state detector 20 in the manner described above.

A preferred embodiment of the present invention has been described above. However, the present invention is not limited to the above embodiment. For example, the solid state detector 20 may be that of the optical readout type. In addition, the X-ray dosage detector 10 may be formed directly on the solid state detector 20 rather than on the resin substrate 15.

1. A mammography apparatus comprising:
a detecting unit constituted by a solid state detector for receiving irradiation of X-rays that bear image information to record the image information, and for outputting image signals that represent the recorded image information, and an X-ray dosage detector, which is provided between a radiation source of the X-rays and the solid state detector, for detecting the dosage of the irradiated X-rays; and
a holding portion for removably holding the detecting unit.

2. A mammography apparatus as defined in claim 1, further comprising:
a grid, for removing scattered radiation, provided between the radiation source of the X-rays and the solid state detector.

3. A detecting unit comprising:
a solid state detector for receiving irradiation of X-rays that bear image information to record the image information, and for outputting image signals that represent the recorded image information;
and an X-ray dosage detector, which is provided between a radiation source of the X-rays and the solid state detector, for detecting the dosage of the irradiated X-rays.

4. A detecting unit as defined in claim 3, further comprising:
a grid, for removing scattered radiation, provided between the radiation source of the X-rays and the solid state detector.
